(19) United States
(12) Patent Application Publication
(10) Pub. No.: US 2005/0239888 A1
(43) Pub. Date: Oct. 27, 2005

(54) Pharmaceutical compositions containing keto-acids for endoperitoneal administration

(76) Inventors: Jose Sebastian Franzone, Torino (IT); Claudio Omini, Bussero (IT); Giuseppe Zuccari, Sant' Angelo Lodigiano (IT)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/522,154

(22) PCT Filed: Jul. 25, 2003
(86) PCT No.: PCT/EP03/08226

§ 371 Date: Jan. 24, 2005

(30) Foreign Application Priority Data
Jul. 26, 2002 (IT) TO2002 A 00672

(51) Int. Cl. A61K 31/4172; A61K 31/198; A61K 31/19

(52) U.S. Cl. 514/557; 514/400; 514/561; 514/566; 514/567

(57) ABSTRACT

Pharmaceutical compositions are described containing ketoaminoacids such as keto-isoleucine, keto-leucine, keto-phenyl alanine, keto-valine and keto-hydroxy-methionine, optionally in association with essential aminoacids and/or vitamins and their use for intraperitoneal administration as a dietary supplement for subjects affected by renal failure or weakness.

The present invention relates to a new use pharmaceutical and in particular to a new way of administration of keto-acids useful as dietary supplements for patients with renal failure.

The emunctory apparatus, of which the kidneys represent an essential element, plays a determining role in the physiological functionality contributing significantly to the maintenance of correct homeostasis of the organism. The kidneys have the function of cleansing from the blood the majority of the products of cellular catabolism and, in particular, the products of catabolism of proteins, which constitutes the majority of the nitrogenous compounds. Renal failure, induced by whatever etiological cause, is characterised by a greater or lesser reduction in the capacity of the kidneys adequately to filter the circulating blood and is characterised by an increase in azotemia. Renal failure can be classified as acute or chronic type. A sharp and often reversible partial or total interruption of the filtration capacity of the kidney, characterised by a substantial reduction in the urinary volume, is classified as acute renal failure (ARF). From the clinical point of view, ARF is associated with a rapid and constant increase of azotemia with the presence or absence of oliguria (<500 ml/per day). The second condition of renal failure is the so-called chronic renal failure (CRF) with various etiopathologies and progressive reduction of the filtrating, capacity of the kidneys. In CRF a progressive distribution of the nephrones is normally observed, which progressively reduces the renal functionality. Cachetia, with loss of both muscular and fat mass, retardation in the growth of children, and diminished protein synthesis are easily observable in those suffering from CRF. Therefore these patients necessitate, in the case of chronic illness, a meticulous attention to the dietetic treatment, as the chronic renal failure gradually develops (1). The correct dietetic dose must be instigated for the purpose of counteracting anorexia, which is one of the early symptoms of this illness. In this context, however, the dose of dietetic protein must be suitably reduced and it has therefore become firmly established that when used in patients with chronic renal failure and in particular those subjected to dialysis, such patients receive an adequate supplement with essential aminoacids. This clinical practice is extremely widespread and numerous scientific studies have attested the validity thereof.

In particular, the use of keto-acids such as keto-isoleucine, keto-leucine, keto-phenyl alanine, keto-valine, etc., which are precursors of essential aminoacids and are directly transformed into corresponding natural aminoacids by the organism after ingestion, have the advantage of reducing the degree of plasmatic urea, of reducing the synthesis of urea and its excretion, and significantly improving the nitrogen balance (2). The aminoacids are traditionally administered to the patient undergoing haemodialysis by venous means with suitable formulations. However, more recently numerous clinical studies (3, 4, 5) have indicated that the oral administration of aminoacids and keto-acids is efficacious as a dietary supplement for patients with renal failure.

Currently there are several commercially available preparations based on keto-acids for use in the oral administration of patients with chronic renal failure. However, these formulations are those traditionally used and in particular take the form of tablets and must be taken even with a posology of ten tablets three times per day. The low practicality and intrinsic difficulty of taking such formulations is entirely evident. The object of the present invention is that of obtaining formulations for intraperitoneal administration and endovenous administration containing keto-acids possibly associated with aminoacids and vitamins as a dietetic supplement for the patient with renal failure or, in general, weakened patients, which are pharmaceutically acceptable and which improve the patient's compliance. It is known in the art that keto-acids administered orally are transformed in the body into corresponding aminoacids by means of a process of transamination effected in part at the cost of non essential aminoacids obtained from the diet and in part with the use of ammonium in the form of ammonia produced by intestinal bacteria.

It is likewise known that the endovenous administration of keto-acids in subjects affected by a deficit of carbamyl phosphate synthetase (6) have shown a rapid increase in the concentration of the respective aminoacids in the serum. The intraperitoneal administration of keto-acids is not known.

Within the scope of the present invention it has been observed, in tests performed on rabbits, that the intraperitoneal administration of a mixture of keto-acids has also caused the appearance, at the serum level, of corresponding aminoacids. Therefore, in correspondence with what is observed for oral administration, the intraperitoneal administration of a mixture of keto-acids has caused a significant increase in the serum of leucine and isoleucine (p<0.02), and valine (p<0.05). It is interesting to observe how the endovenous administration of keto-acids causes a rapid plasmatic peak of corresponding aminoacids, but that this becomes exhausted equally rapidly because of the rapid incorporation of these into the protein structures. As opposed to endovenous administration and very much more advantageously, the intraperitoneal administration of keto-acids causes a plasmatic concentration of the corresponding aminoacids more slowly to start with but over a more extended time. This action is evidently an advantage with respect to the endovenous method of administration. In fact, the introperitoneal administration of the keto-acids makes possible a more protracted temporal use of the compounds in a manner similar to that of oral administration, which has been shown to have been effective in dialysed subjects, whilst preventing the already described disadvantages of the necessity for taking up to ten tablets several times a day. It is in fact an entirely surprising result that the single intraperitoneal administration of a solution of keto-acids, such as that hereinafter reported, has made it possible to obtain a sufficient daily quantity of aminoacid supplementation in dialysed patients. It is moreover important to underline how the use of keto-acids in association with essential aminoacids leads to a consistent improvement in efficacy and therefore in the present invention there are likewise preferred the formulations suited to intraperitoneal administration comprising the association of keto-acid and aminoacids.

The subject of the invention is defined by the claims which follow.

For the single purpose of better representing the present invention the following examples of inventive formulations, with an indication of the usable dosage interval, are provided hereinafter.

### EXAMPLE 1

100 g of formulation containing:
keto-isoleucine 0.1-1.9 g;
keto-leucine 0.1-2.2 g;
keto-valine 0.30-2.10 g;
keto-hydroxy-methionine 0.1-1.5 g;
L-phenyl-alanine 0.10-1.90 g;
L-lysine 0.5-2.5 g;
L-threonine 0.2-2.0 g;
L-histidine 0.1-1.0 g;
L-tyrosine 0.01-0.2 g;
HCl 37% q.s. (as needed) to pH 7.0+/−0.2;
Na metabisulphite 0.05 g;
water for injectable preparations q.s. (as needed) to 100

### EXAMPLE 2

1000 ml of formulation containing:
Ca keto-isoleucine 0.3-2.9 g
Ca keto-leucine 0.1-3.2 g
Ca keto-valine 0.5-4.1 g
Ca keto-hydroxy-methionine 0.1-1.15 g
L-phenyl-alanine 0.1-1.5 g
L-lysine 0.5-2.5 g
L-threonine 0.2-2.0 g
L-histidine 0.1-1.5 g
L-tyrosine 0.01-1.0 g
L-serine 0.05-1.5 g
L-tryptophan 0.05-1.0 g
L-alanine 0.05-2.5 g
L-arginine 0.3-2.5 g
Glycine 0.03-1.5 g
L-proline 0.1-1.5 g
Na lactate 2.0-8.0 g
NaCl 2.0-10 g
MgCl₂ 0.01-1 g
HCl q.s. to pH 6.5-7.0
H₂O q.s. to 1000 ml

These formulations are easily obtainable by one skilled in the art, possibly referring to texts in use in the pharmaceutical field. These formulations are liquid and stable over time; moreover, since the formulations are easily dispersible cold in water or other aqueous liquids suitable for endovenous or intraperitoneal administration in man, such as for example a physiological solution, glucosate solution etc, the injectable formulation can easily be prepared even extemporaneously and immediately before use. In the above cited example sodium metabisulphite was utilised as preservative agent, however other substances normally used for the preservation of injectable pharmaceutical products can likewise be utilised.

The association of vitamins, in particular of group B, is likewise within the scope of the present invention in that the necessity to supplement the dialysed subject with poly-vitamin preparations is known in the art. Therefore the use of formulations such as that indicated above in association with water-soluble poly-vitamin complexes is entirely straightforward and represents an undeniable advantage of the present invention. Moreover, if required, salts of usable keto-acids can be based on Ca or other cations or possibly the formulation can be added to specific water-soluble salts to comply with the patient's requirements.

Solely for the purpose of better further representing the formulations described hereinabove in the present invention the following specific example is provided.

### EXAMPLE 3

100 g of formulation containing:
salts of calcium of: keto-isoleucine 0.5 g, keto-leucine 1.0 g;
keto-valine 0.8 g;
keto-hydroxy-methionine 0.4 g;
L-phenyl alanine 0.40 g;
L-lysine 1.0 g;
L-threonine 1.0 g;
L-histidine 0.4 g;
L-tyrosine 0.03 g;
vitamin B1 0.01 g;
vitamin B2 0.005 g;
vitamin B6 0.004 g;
nicotinamide 0.04 g;
D-panthothenol 0.006 g;
vitamin B12 8 mcg;
biotin 500 mcg;
HCL 37% as necessary pH 7.0+/−0.2;
Na metabisulphite 0.05 g;
water for injectable preparations as necessary to 100 g.

### BIBLIOGRAPHY

1. Merck manual of diagnosis and therapy 2^{nd} Italian edition 1990
2. Fynn E S et al; Am. J. Clin. Nutr. 1978; 31(10): 1776-83
3. Ulm A. et al; Am. J. Clin. Nutr. 1978; 31(10): 1827-30
4. Hecking E. et al; Z Emahrungswiss 1982; 21(4): 299-311
5. Dalton R N & Chantler C.; Kidney Int. Suppl. 1983; 15: S11-5
6. Batshaw M et al; N. Eng. J. Med. 1975; 292: 1085-90

1. A pharmaceutically acceptable composition for intraperitoneal administration wherein said composition comprises containing keto-acids and their pharmaceutically acceptable salts in amounts effective to act as a dietary supplement for patients with renal failure or weakness.

2. A composition according to claim 1 containing keto-acids selected from the group consisting of keto-isoleucine, keto-leucine, keto-phenyl alanine, keto-valine, keto-hydroxy-methionine and mixtures thereof.

3. A composition according to claim 1 containing a keto-acid selected from the following group, in an amount within the indicated concentration range:
keto-isoleucine 0.1-1.9 g;
keto-leucine 0.1-2.2 g;
keto-valine 0.30-2.10 g;
keto-hydroxy-methionine 0.1-1.5 g;
and their mixtures for 100 g of final formulation.

4. A composition according to claim 1, containing keto-acids in association with aminoacids.

5. A composition according to claim 4, containing an aminoacid selected from the following group, in an amount within the indicated concentration range:
L-phenyl-alanine 0.10-1.90 g;
L-lysine 0.5-2.5 g;
L-threonine 0.2-2.0 g;
L-histidine 0.1-1.0 g;
L-tyrosine 0.01-0.2 g;
and their mixtures to 100 g of final formulation.

6. A composition according to claim 4, containing aminoacids selected from the following group, in an amount within the indicated concentration range:
L-phenyl-alanine 0.1-1.5 g
L-lysine 0.5-2.5 g
L-threonine 0.2-2.0 g
L-histidine 0.1-1.5 g
L-tyrosine 0.01-1.0 g
L-serine 0.05-1.5 g
L-tryptophan 0.05-1.0 g
L-alanine 0.05-2.5 g
L-arginine 0.3-2.5 g
Glycine 0.03-1.5 g
L-proline 0.1-1.5 g
and their mixtures to 1000 ml of final formulation.

7. A composition according to claim 6 containing the following compounds, in an amount within the indicated concentration range:
Na lactate 2.0-8.0 g
NaCl 2.0-10 g
MgCl₂ 0.01-1 g
HCl q.s. to pH 6.5-7.0
H₂O q.s. 1000 ml

8. A composition according to claim 1 containing keto-acids, aminoacids and vitamins.

9. A composition according to claim 8 containing water-soluble vitamins of group B.

10. A composition according to claim 9, containing vitamins selected from the group consisting of vitamin B1, vitamin B2, vitamin B6, vitamin B12, nicotinamide, D-panthothenol and mixtures thereof.

11. A composition according to claim 1, containing salts with cations of said keto-acids, optionally with added inorganic and organic salts.

12. A composition according to claim 1, dissolved in an aqueous solution suitable for parenteral administration.

13. A composition according to claim 1 in packaging containing multi-dose, mono-dose, daily mono-dose ready for use or alternatively to be dissolved ready for use.

14. A method for providing a patient with renal failure or weakness with an amino acid dietary supplement, said method comprising administering a pharmaceutically effective amount of a composition according to claim 1 by intraperitoneal administration.
